(19) United States
(12) Patent Application Publication
(10) Pub. No.: US 2016/0082066 A1
(43) Pub. Date: Mar. 24, 2016

(54) Method Use of Polymethoxyflavones (PMFs) in Body Composition Management

(71) Applicants: Vladimir Badmaev, Staten Island, NY (US); Min-Hsiung Pan, Kaohsiung City (TW)

(72) Inventors: Vladimir Badmaev, Staten Island, NY (US); Min-Hsiung Pan, Kaohsiung City (TW)

(21) Appl. No.: 14/495,840

(22) Filed: Sep. 24, 2014

(51) Int. Cl. A61K 36/77; A61K 31/353

(52) U.S. Cl. CPC A61K 36/77; A61K 31/353

(57) ABSTRACT

Synergistic composition of Citrus sp. polymethoxyflavones (PMFs) with catechins of tea sp. and Lychee fruits in body weight management application.

### FIELD OF INVENTION

The present invention is related to method of use of polymethoxyflavones (PMFs) from Citrus sp., catechins from tea sp. and the whole Lychee fruit to prevent lipogenesis, adipogenesis, adiposity, overweight and obesity with food or food supplement applications.

### BACKGROUND OF INVENTION

The consensus among health professionals supports a growing trend for safe, non-metabolic stimulant based body weight management. The new approach takes into consideration a meaningful lifestyle changes to sustain weight loss obtained with a non-stimulant nutritional foods and food supplements. The new class of botanical weight loss compounds would effectively and safely prevent adipogenesis due to an excessive calorie intake and/or due to the age-related metabolic deterioration and slow-down.

As caloric intake increases and/or metabolic rate declines in the aging process, adipocytes accumulate triacylglycerols (triglycerides) for the energy reserves, resulting in increased adipogenesis or adipose tissue mass, and consequently adiposity, overweight and obesity. In addition to fat storage, adipose tissue is a major endocrine and metabolic organ by secretion of adipocytokines, cytokines, growth factors and hormones involved in host immunity, energy homeostasis, systemic insulin sensitivity and tissue regeneration. In overweight and obesity, the excessive adipose tissue contributes to abnormal cytokine and hormone production and metabolic dysfunction (1).

The essential step leading to adipogenesis depends on differentiation of immature adipocytes or pre-adipocyte cells into mature adipocytes with subsequent increase in body fat mass. In the in vitro tissue culture, differentiation of preadipocytes results in morphological and biochemical changes including reentry into the cell cycle for an additional two rounds of cell division, mitotic clonal expansion (MCE), and terminal differentiation to mature adipocytes followed by changes in genetic program for increase in lipid synthesis and storage (2).

This process is controlled by a set of transcription factors such as CCAAT/enhancer-binding proteins (C/EBPs), peroxisome proliferator-activated receptor PPARgamma, sterol regulatory element-binding protein (SREBP)-1c, and cellular signaling cascade involved in the cell cycle and insulin-dependent signaling pathways. These transcription factors play critical role in early phase of adipogenesis (3). In addition, activation of the PPARgamma promotes adipose differentiation in the fibroblasts (4). Insulin and insulin-like growth factor-1 (IGF-1) also increases the rate of adipocyte differentiation (5). The AMP activated protein kinase (AMPK) acts as a nutrient sensor and central regulator of cellular energy homeostasis. The activated AMPK leads to inhibition of adipocyte differentiation and decreased adipogenesis (6, 7). Phosphorylation of metabolic enzyme acetyl-CoA carboxylase 1 (ACC1) and HMG-CoA reductase (HMGCR) by AMPK promotes fatty acid oxidation and reduces cholesterol synthesis (8, 9).

The above discussed biological steps leading to adipogenesis are currently targeted in prevention of overweight and obesity conditions caused by excess calorie intake and/or age-related process of metabolic decline. The mechanisms of adipocyte differentiation and regulation of adipogenesis have been utilized in the invention's anti-overweight and obesity strategy. There are three botanicals regulating adipogenesis discussed in the invention: Citrus sp. (the peel), Camellia sinensis (the green tea) and Litchi sinensis (the fruits of Lychee).

Citrus sp. peel flavonoids and polymethoxyflavone, nobiletin, have recently been discussed in literature as compounds potently suppressing the differentiation of tissue culture 3T3-L1 preadipocytes into adipocytes, alleviating obesity and insulin resistance in a high-fat diet-induced obesity in mice (10,11). Hydroxylated polymethoxyflavones (HPMs) isolated from Citrus sp. peel have similar biological properties to polymethoxyflavones (PMFs).

Catechins contained in Camellia sinensis or green tea are believed to play a role in activating body metabolism, which may lead to the weight loss. The epigallo-catechin gallates or EGCG, the most bioactive catechin in green tea inhibits catechol-O-methyltransferase, an enzyme involved in biodegradation of catecholamines. This mechanism via the catecholamine-mediated stimulation of β-adrenergic receptors has been hypothesized to enhance the metabolic rate and be conductive to the weight loss. However, the clinical studies showed that EGCG stand alone would not increase the resting metabolic rate (RMR) and the thermal effects of feeding (TEF) in the population of young, healthy adults (12). The outcome of number of randomized controlled trials (RCTs) evaluating the potential role of green tea in weight loss indicate that green tea either produces no weight loss effects or induces a small, statistically non-significant weight loss in overweight or obese adults (13).

The Lychee (Lytchi sinensis) is a fruit with high content of vitamin C, approximately 72 mg of vitamin C per 100 grams of fruit (USDA. “Litchis, raw”. USDA. Retrieved 5 Apr. 2013). On average nine Lychee fruits would meet an adult's daily recommended Vitamin C requirement. Lychee fruit (one cup) also provides, several essential trace elements including 14% Daily Value (DV) of copper, 9% DV of phosphorus, and 6% DV of potassium (for a 2000-calorie diet) (14). Lychees have moderate amounts of phenolics e.g. flavan-3-ol monomers and dimers representing about 87.0% of the fruit's phenolic compounds. The cyanidin-3-glucoside is a major anthocyanin and represents 91.9% of anthocyanins in fruit's composition. In addition, the lychee fruit also contains small amounts of malvidin-3-glucoside (15). Lychee fruits do not have a known role regulating lipogenesis, adipogenesis or metabolic functions related to body weight management.

### DESCRIPTION OF INVENTION

The invention provides a food and/or nutriceutical composition for reducing and preventing lipogenesis, adipogenesis, adiposity, overweight and obese conditions. The invention is outcome of an unexpected synergy involving phytochemicals found in Citrus species peel, green tea and Lychee fruit. Among the phytochemicals of invention, polymethoxyflavones (PMFs), particularly in the peel of sweet oranges (Citrus sinensis) and mandarin oranges (Citrus reticulate), have been demonstrated as principle compounds of invention synergistically and significantly enhanced by addition of green tea extract and Lychee fruits.

The Synergy of Invention's PMFs with ECG, EGCG

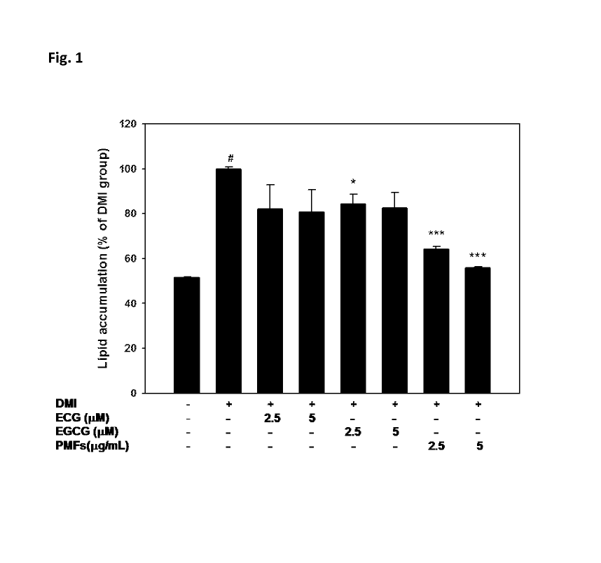

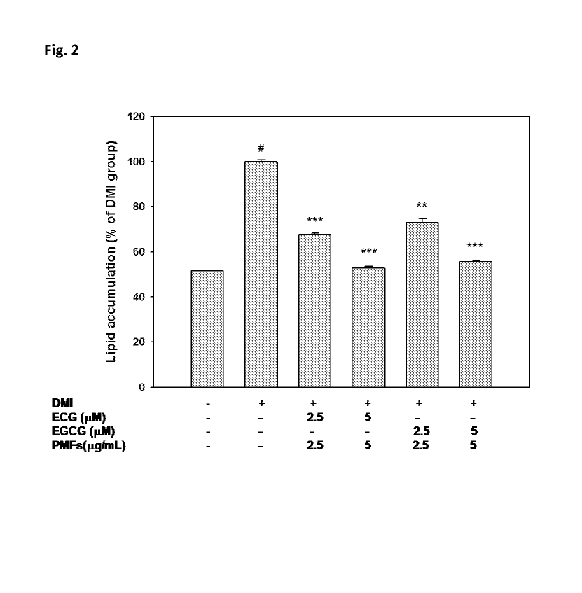

To compare the effects of green tea extracted epicatechin gallates (ECG), epigallocatechin gallates (EGCG) and PMFs on adipocyte differentiation, 3T3-L1 pre-adipocytes were treated with the differentiation culture medium and insulin (DMI) with or without ECG, EGCG and PMFs. Ten days after the initiation of differentiation, lipid accumulation was measured by Oil Red O staining. The results showed that PMFs significantly and dose dependently reduced lipid accumulation in differentiated adipocytes, whereas the presence of ECG or EGCG alone decreased only slightly lipid accumulation in differentiated adipocytes (FIG. 1). However, the ECG or EGCG combined with PMFs synergistically inhibited lipid accumulation at statistically significantly higher levels as compared to PMFs or ECG or EGCG standalone (FIG. 2).

FIG. 1. Inhibitory effect of ECG, EGCG and PMFs on lipogenesis and adipogenesis in 3T3-L1preadipocytes. 3T3-L1 preadipocytes were incubated with DMI (DMEM with IBMX, DEX and insulin) for two days, and then replaced with DMEM medium containing insulin with or without ECG, EGCG and PMFs at indicated concentrations, respectively for 8 days. The 3T3-L1preadipocytes were stained with Oil Red O and photographed. Lipid content was extracted from Oil Red O stained cells by 2-propanol and quantified with spectrophotometric analysis at 520 nm. Data were presented as mean±SE and each experiment was independently performed three times with similar results. ^{#}P<0.001 indicates statistically significant differences from FCS-treated group. *P<0.05 and ***P<0.001 were compared with DMI-treated alone group.

FIG. 2. Synergistic effect of combined ECG or EGCG with PMFs on inhibition of lipogenesis and adipogenesis in 3T3-L1 preadipocytes. 3T3-L1 preadipocytes were incubated with DMI (DMEM with IBMX, DEX and insulin) for two days, and then replaced with DMEM medium containing insulin with or without ECG, EGCG and PMFs at indicated concentrations, respectively for 8 days. The 3T3-L1preadipocytes were stained with Oil Red 0. Lipid content was extracted from Oil Red O stained cells by 2-propanol and quantified with spectrophotometric analysis at 520 nm. Data were presented as mean±SE and each experiment was independently performed three times with similar results. ^{#}P<0.001 indicates statistically significant differences from FCS-treated group. **P<0.05 and ***P<0.001 were compared with DMI-treated alone group.

The Synergy of Invention's PMFs with Lychee Fruits

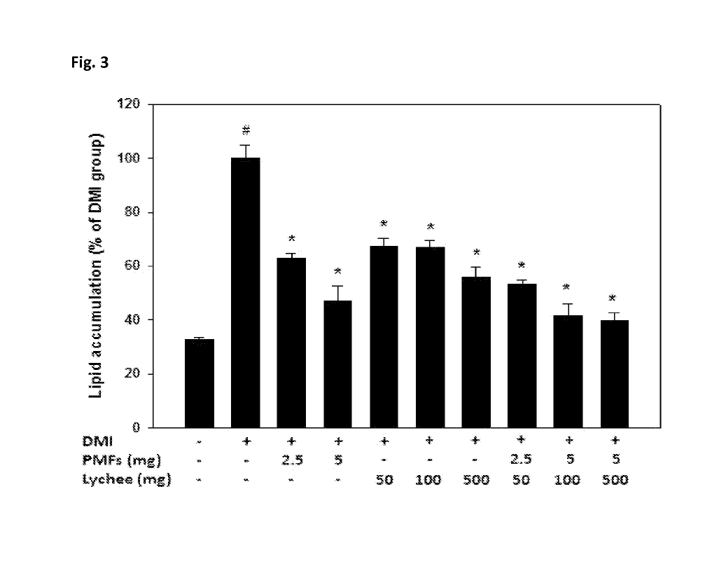

The 3T3-L1 adipocytes treated with the DMI, Lychee or PMFs showed significantly less lipid accumulation demonstrated by the Oil-Red O staining (FIG. 3). The combination of Lychee fruits and PMFs showed the additional and dose dependent increase of lipid accumulation as compared to adipocytes treated with Lychee or PMFs standalone (FIG. 3).

FIG. 3. Synergistic effect of combined Lychee fruits and PMFs on inhibition of lipogenesis and adipogenesis in 3T3-L1preadipocytes. 3T3-L1 preadipocytes were incubated with DMI (DMEM with IBMX, DEX and insulin) for two days, and then replaced with DMEM medium containing insulin with or without Lychee extracts and PMFs, or in combination at indicated concentrations, respectively for 8 days. The 3T3-L1preadipocytes were stained with Oil Red O and photographed. Lipid content was extracted from Oil Red O stained cells by 2-propanol and quantified with spectrophotometric analysis at 520 nm. Data were presented as mean±SE and each experiment was independently performed three times with similar results. ^{#}P<0.001 indicates statistically significant differences from FCS-treated group. *P<0.001 compared with DMI-treated alone group.

### THE SYNERGY OF COMPOSITION OF INVENTION: PMFS, LYCHEE FRUITS AND ECG/EGCG (GREEN TEA EXTRACT)

In the in vitro experiments, treatment of 3T3-L1 adipocytes with composition of invention, i.e. PMFs, Lychee fruits and ECG or/and EGCG from green tea resulted in a significantly higher inhibition of lipid accumulation in the adipocytes as compared to the results obtained with the individual components of invention (FIG. 2 and FIG. 3).

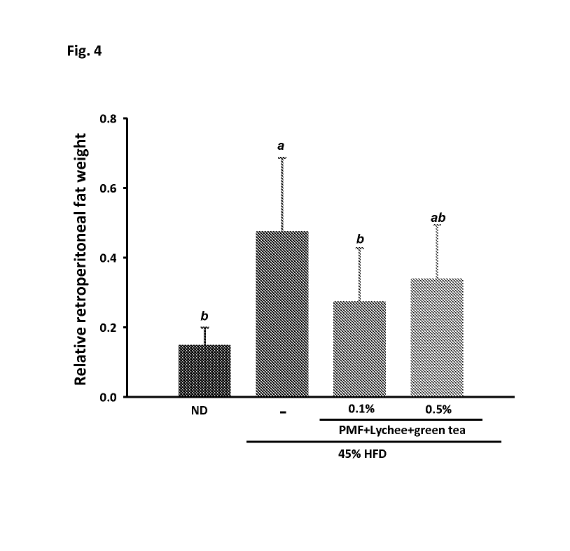

In addition, composition of invention, i.e. 0.1 and 0.5% supplements respectively, prevented high-fat diet-induced obesity in mice. The 8 week supplementation of C57BL/6 mice with 45% high fat diet (HFD) with PMFs, Lychee fruits and ECG/EGCG (green tea extract) resulted in a statistically significant decrease in body weight (Table 1) and retroperitoneal fat (FIG. 4) as compared to mice receiving 45% HFD diet stand alone. There was no significant change in levels of food intake between HFD diet group and the composition of invention intake, however normal diet group had statistically significantly higher food intake than the composition of invention groups. The composition of invention supplemented for 8 weeks at 0.1% and 0.5% concentrations resulted in statistically significantly higher levels of weight loss and retroperitoneal fat loss compared to the group of mice receiving 45% HFD diet stand alone. The experimental group receiving composition of invention at 0.5% has been showing slight advantage in body weight loss over the 0.1% group and based on internal discussion of the results the higher concentration could over time result in statistically higher weight loss comparing to the lower concentration group. In addition, supplementation with composition of invention as compared to the HFD group statistically significantly lowered total cholesterol, increased high density lipoproteins, lowered liver enzymes GOT and GPT and showed trend to lower levels of triglycerides indicating positive effects on body lipids metabolism and homeostasis (Table 2).

In conclusion, we demonstrated in in vitro and preclinical experiments with rodents on HFD diet synergistic action of composition of invention lowering body weight, decreasing body fat and improving body metabolism. Addition of ECG/EGCG and Lychee fruits significantly and synergistically potentiate the PMFs' attenuation and prevention of lipogenesis, adipogenesis, adiposity and overweight and obese conditions.

Table 1. Effects of PMFs, Lychee fruits and green tea formula on body weight gain and food Intake in mice fed with high fat diet (HFD). Mice were fed experimental diets for 8 weeks and the body weight and food intake were monitored twice weekly. The average body weight of each group is expressed as the mean±SE (n=6 per group), and statistical analysis was done by one-way ANOVA and Duncan's Multiple Range Test and results were indicated by different letters a, b, c. ND, normal diet; HFD, high-fat diet. Means with different letters are significantly different (Duncan's multiple range test at P<0.05).

Table 2. Effects of PMFs, Lychee fruits and green tea formula on serum biochemical parameters in mice fed with high fat diet (HFD). Mice were fed HFD supplemented with or without PMF, Lychee and green tea (0.1 and 0.5%) for 8 weeks. The activities of serum GOT, GPT, TG and T-cho were analyzed. Data are presented as the mean±SE (n=6 per group), and statistical analysis was done by one-way ANOVA and Duncan's Multiple Range Test and results were indicated by different letters a, b, c. ND, normal diet; HFD, high-fat diet. Means with different letters are significantly different (Duncan's multiple range test at P<0.05).

FIG. 4. Effect of PMFs, Lychee fruits and green tea formula on relative adipose tissue weight in HFD-fed C57BL/6 mice. Mice were fed HFD diet supplement with or without PMF, Lychee and green tea formula (0.1 and 0.5%) for 8 weeks. The relative retroperitoneal fat weight was expressed as a percentage of body weight (adipose tissue weight/body weight×100). Data are expressed as the mean±SE, and statistical difference was analyzed using one-way ANOVA. ND, normal diet; HFD, high-fat diet. Means with different letters are significantly different (Duncan's multiple range test at P<0.05).

Methods of Manufacture of Invention

The manufacture of invention is based on principles of environmental responsibility preferably utilizing green technology without or with minimal application of environmentally harmful solvents, CO₂ supercritical extraction and adiabatic extraction methods. The Citrus sp. peel extract can be standardized for 20-90% of PMFs. Green tea extract can be standardized preferably to individual or total polyphenols, e.g. 40-95% catechins and Lychee fruit can be standardized preferably to flavan-3-ol monomers (cyanidin-3-glucoside) and dimers of the whole dried fruit. The composition of invention is blended to obtain food grade, water soluble, water dispersible and cosmetic grades products with preferred ratio of ingredients in 1:1:1 proportion. The composition of invention maybe adjusted as needed to obtain optimal nutritional and nutriceutical products for their weight management and organoleptic properties.

TABLE 1

| | | | 45% HFD + | 45% HFD + |
|---|---|---|---|---|
| | | | 0.1% PMF + | 0.5% PMF + |
| | | | 0.1% Lychee + | 0.5% Lychee + |
| | Normal diet | 45% HFD | 0.1% green tea | 0.5% green tea |
| Initial wt | 21.3 ± 1.3 | 21.1 ± 1.2 | 20.9 ± 1.3 | 21.8 ± 1.0 |
| (g) | | | | |
| Final wt | 26.3 ± 0.9^{b} | 29.6 ± 1.5^{a} | 26.9 ± 2.2^{b} | 26.6 ± 1.5^{b} |
| (g) | | | | |
| Wt gain | 5.1 ± 2.1^{b} | 9.1 ± 2.2^{a} | 5.4 ± 2.0^{b} | 4.6 ± 1.9^{b} |
| (g) | | | | |
| Food | 5.4 ± 0.1^{a} | 3.7 ± 0.1^{bc} | 3.9 ± 0.2^{b} | 3.7 ± 0.1^{c} |
| intake | | | | |
| (g/mouse/ | | | | |
| day) | | | | |

TABLE 2

| | | | 45% HFD + | 45% HFD + |
|---|---|---|---|---|
| | | | 0.1% PMF + | 0.5% PMF + |
| | | | 0.1% Lychee + | 0.5% Lychee + |
| Activity | Normal diet | 45% HFD | 0.1% green tea | 0.5% green tea |
| GOT (U/L) | 64.00 ± 20.80^{c} | 224.67 ± 66.34^{a} | 79.00 ± 23.81^{c} | 154.33 ± 44.88^{b} |
| GPT (U/L) | 21.33 ± 4.23 | 21.33 ± 6.12 | 17.33 ± 9.81 | 28.33 ± 12.01 |
| TG (mg/dL) | 140.67 ± 58.67 | 125.83 ± 38.23 | 95.83 ± 23.71 | 113.17 ± 36.89 |
| T-cho (mg/dL) | 86.00 ± 18.63^{b} | 140.67 ± 15.51^{a} | 127.17 ± 31.29^{a} | 121.33 ± 12.21^{a} |
| HDL (mg/dL) | 67.67 ± 19.16^{b} | 102.00 ± 50.91^{ab} | 123.33 ± 49.47^{a} | 122.33 ± 21.22^{a} |

### Specifications of Invention

### REFERENCE LIST

1. Guilherme, A.; Virbasius, J. V.; Puri, V.; Czech, M. P. Adipocyte dysfunctions linking obesity to insulin resistance and type 2 diabetes. Nat. Rev. Mol. Cell Biol. 2008, 9 (5), 367-377.
2. Rosen, E. D.; MacDougald, O. A. Adipocyte differentiation from the inside out. Nat. Rev. Mol. Cell Biol. 2006, 7 (12), 885-896.
3. Cao, Z.; Umek, R. M.; McKnight, S. L. Regulated expression of three C/EBP isoforms during adipose conversion of 3T3-L1 cells. Genes Dev. 1991, 5 (9), 1538-1552.
4. Tontonoz, P.; Hu, E.; Spiegelman, B. M. Stimulation of adipogenesis in fibroblasts by PPAR gamma 2, a lipid-activated transcription factor. Cell 1994, 79 (7), 1147-1156.
5. Xu, J.; Liao, K. Protein kinase B/AKT 1 plays a pivotal role in insulin-like growth factor-1 receptor signaling induced 3T3-L1 adipocyte differentiation. J. Biol. Chem. 2004, 279 (34), 35914-35922.
6. Giri, S.; Rattan, R.; Haq, E.; Khan, M.; Yasmin, R.; Won, J. S.; Key, L.; Singh, A. K.; Singh, I. AICAR inhibits adipocyte differentiation in 3T3L1 and restores metabolic alterations in diet-induced obesity mice model. Nutr. Metab (Lond) 2006, 3, 31.
7. Dagon, Y.; Avraham, Y.; Berry, E. M. AMPK activation regulates apoptosis, adipogenesis, and lipolysis by elF2alpha in adipocytes. Biochem. Biophys. Res. Commun. 2006, 340 (1), 43-47.
8. Hardie, D. G. Regulation of fatty acid and cholesterol metabolism by the AMP-activated protein kinase. Biochim. Biophys. Acta 1992, 1123 (3), 231-238.
9. Lee, W. J.; Kim, M.; Park, H. S.; Kim, H. S.; Jeon, M. J.; Oh, K. S.; Koh, E. H.; Won, J. C.; Kim, M. S.; Oh, G. T.; Yoon, M.; Lee, K. U.; Park, J. Y. AMPK activation increases fatty acid oxidation in skeletal muscle by activating PPARalpha and PGC-1. Biochem. Biophys. Res. Commun. 2006, 340 (1), 291-295.
10. Kim, G. S.; Park, H. J.; Woo, J. H.; Kim, M. K.; Koh, P. O.; Min, W.; Ko, Y. G.; Kim, C. H.; Won, C. K.; Cho, J. H. Citrus aurantium flavonoids inhibit adipogenesis through the Akt signaling pathway in 3T3-L1 cells. BMC. Complement Altern. Med. 2012, 12, 31.
11. Lee, Y. S.; Cha, B. Y.; Choi, S. S.; Choi, B. K.; Yonezawa, T.; Teruya, T.; Nagai, K.; Woo, J. T. Nobiletin improves obesity and insulin resistance in high-fat diet-induced obese mice. J. Nutr. Biochem. 2013, 24 (1), 156-162.
12. Lonac M C, Richards J C, Schweder M M, Johnson T K, Bell C. Influence of short-term consumption of the caffeine-free, epigallocatechin-3-gallate supplement, Teavigo, on resting metabolism and the thermic effect of feeding. Obesity 2011 February; 19(2):298-304. doi: 10.1038/oby.2010.181. Epub 2010 Aug. 19.
13. Jurgens T M, Whelan A M, Killian L, Doucette S, Kirk S, Foy E. Green tea for weight loss and weight maintenance in overweight or obese adults. Cochrne Database Syst. Rev. 2012 Dec. 12; 12:CD008650. doi: 10.1002/14651858.CD008650.pub2.
14. Pierre Brat, Stéphane Georgé, Annick Bellamy, Laure Du Chaffaut, Augustin Scalbert, Louise Mennen, Nathalie Arnault and Marie Josèphe Amiot (9 2006). “Daily Polyphenol Intake in France from Fruit and Vegetables”. The Journal of Nutrition 136 (9): 2368-2373. PMID 16920856.
15. Changes in phenolic compounds in Litchi (Litchi chinensis Sonn.) fruit during postharvest storage. Donglin Zhang, Peter C. Quantick and John M. Grigor, Postharvest Biology and Technology, Volume 19, Issue 2, June 2000, Pages 165-172 doi:10.1016/S0925-5214(00)00084-3.

We claim:

1. A method of promoting optimal body composition in an individual in need thereof, comprising administering to the individual an effective amount of PMFs, ECG, EGCG from tea sp. and Lychee fruits.

2. A method of preventing lipogenesis in an individual in need thereof, comprising administering to the individual an effective amount of food or food supplement composition in claim 1.

3. A method of preventing adipogenesis in an individual in need thereof, comprising administering to the individual an effective amount of food or food supplement composition in claim 1.

4. A method of preventing adiposity in an individual in need thereof, comprising administering to the individual an effective amount of food or food supplement composition in claim 1.

5. A method of preventing overweight in an individual in need thereof, comprising administering to the individual an effective amount of food or food supplement composition in claim 1.

6. A method of preventing obesity in an individual in need thereof, comprising administering to the individual an effective amount of food or food supplement composition in claim 1.

7. The method of claim 1 wherein the PMFs are administered in a preferred daily dose of 300-1000 mg.

8. The method of claim 1 wherein the ECG, EGCG is administered in a preferred daily dose of 100-300 mg.

9. The method of claim 1 wherein the Lychee fruit is administered in a preferred daily dose of 500-1500 mg.

10. The method of claim 1 wherein preferred ratio of PMFs, catechins from tea sp. and Lychee fruit is 1:1:1.

11. The method of claim 1 wherein PMFs can be substituted with hydroxylated polymethoxyflavones (HPMs).
